(19) United States
(12) Patent Application Publication
(10) Pub. No.: US 2008/0177308 A1
(43) Pub. Date: Jul. 24, 2008

(54) POLYMERIC JOINT COMPLEX AND METHODS OF USE

(75) Inventor: Thomas J. McLeer, Redmond, WA (US)

Correspondence Address: SHAY GLENN LLP, 2755 CAMPUS DRIVE, SUITE 210, SAN MATEO, CA, 94403, US

(73) Assignee: Archus Orthopedics, Inc., Redmond, WA (US)

(21) Appl. No.: 11/934,720

(22) Filed: Nov. 2, 2007

Related U.S. Application Data
(63) Continuation of application No. 11/244,420, filed on Oct. 4, 2005.
(60) Provisional application No. 60/616,093, filed on Oct. 4, 2004.

(51) Int. Cl. A61B 17/58

(52) U.S. Cl. 606/247; 606/280

(57) ABSTRACT

The invention describes a variety of implantable artificial joint complexes adapted for implantation within a target joint space within a human body. The joint complexes comprise: an expandable joint segment adapted to fit within the target joint space; and at least one of a first cannulated anchor adapted to engage the expandable joint segment and adapted to engage a bony structure adjacent the target joint space; and a second anchor adapted to engage the expandable joint segment and adapted to engage a bony structure adjacent a target joint space. The invention also discloses methods of implanting a patient specific artificial joint complex. The methods include the steps of: accessing a target joint space by creating an access hole through an adjacent bony structure; inserting a joint complex device having a cannulated anchor and an expandable joint segment through the access hole with the expendable joint segment being positioned between the surfaces forming the joint; injecting material into the expandable joint segment; and sealing access to the target joint space.

### CROSS-REFERENCE

This application is a continuation of U.S. patent application Ser. No. 11/244,420, filed Oct. 4, 2005, which claims the benefit of U.S. Provisional Patent Application Ser. No. 60/616,093 to Thomas J. McLeer, filed Oct. 4, 2004, and entitled “Polymer Joint Complex”, which is also incorporated herein by reference.

### FIELD OF THE INVENTION

This invention relates to implantable spinal devices, systems, and methods for treating various types of spinal pathologies. The invention relates in particular to a polymeric facet joint complex providing a flexible artificial joint complex.

### BACKGROUND OF THE INVENTION

Back pain, particularly in the small of the back, or lumbosacral region (L4-S1) of the spine, is a common ailment. In many cases, the pain severely limits a person's functional ability and quality of life. Back pain interferes with work, routine daily activities, and recreation. It is estimated that Americans spend $50 billion each year on low back pain alone. It is the most common cause of job-related disability and a leading contributor to missed work.

Through disease or injury, the laminae, spinous process, articular processes, facets and/or facet capsule(s) of one or more vertebral bodies along with one or more intervertebral discs can become damaged which can result in a loss of proper alignment or loss of proper articulation of the vertebra. This damage can result in anatomical changes, loss of mobility, and pain or discomfort. For example, the vertebral facet joints can be damaged by traumatic injury or as a result of disease. Diseases damaging the spine and/or facets include osteoarthritis where the cartilage of joint is gradually worn away and the adjacent bone is remodeled, ankylosing spondylolysis (or rheumatoid arthritis) of the spine which can lead to spinal rigidity, and degenerative spondylolisthesis which results in a forward displacement of the lumbar vertebra on the sacrum. Damage to facet joints of the vertebral body often can also results in pressure on nerves, commonly referred to as “pinched” nerves, or nerve compression or impingement. The result is pain, misaligned anatomy, and a corresponding loss of mobility. Pressure on nerves can also occur without facet joint pathology, e.g., a herniated disc.

One conventional treatment of facet joint pathology is spine stabilization, also known as intervertebral stabilization. Intervertebral stabilization desirably controls, prevents or limits relative motion between the vertebrae, through the use of spinal hardware, removal of some or all of the intervertebral disc, fixation of the facet joints, bone graft/osteo-inductive/osteo-conductive material (with or without concurrent insertion of fusion cages) positioned between the vertebral bodies, and/or some combination thereof, resulting in the fixation of (or limiting the motion of) any number of adjacent vertebrae to stabilize and prevent/limit/control relative movement between those treated vertebrae. Stabilization of vertebral bodies can range from the insertion of motion limiting devices (such as intervertebral spacers, artificial ligaments and/or dynamic stabilization devices), through insertion of devices promoting arthrodesis (rod and screw systems, cable fixation systems, fusion cages, etc.), up to and including complete removal of some or all of a vertebral body from the spinal column (which may be due to extensive bone damage and/or tumorous growth inside the bone) and insertion of a vertebral body replacement (generally anchored into the adjacent upper and lower vertebral bodies). Various devices are known for fixing the spine and/or sacral bone adjacent the vertebra, as well as attaching devices used for fixation, including: U.S. Pat. Nos. 4,611,581; 4,805,602; 5,129,900; 5,474,555; 5,569,247; 5,575,792; 5,643,263; 5,683,392; 5,688,274; 5,690,630; 5,725,527; 5,738,585; 5,741,255; 5,782,833; 5,797,911; 5,863,293; 5,879,350; 5,885,285; 5,891,145; 5,964,760; 6,010,503; 6,019,759; 6,022,350; 6,074,391; 6,077,262; 6,090,111; 6,132,430; 6,248,105; 6,290,703; 6,451,021; 6,471,705; 6,520,963; 6,524,315; 6,540,749; 6,547,790; 6,554,843; 6,565,565; 6,619,091; 6,638,321; 6,811,567; and U.S. Patent Publication No. 2002/0120272; 2002/0085912; and 2005/0177240.

### SUMMARY OF THE INVENTION

Moreover, there is a need in the art for methods and devices which facilitate the less-invasive, minimally-invasive and/or non-invasive correction, restoration, or augmentation of the anatomical characteristics (including size, shape, orientation and/or relationship) of anatomical features of joints such as the facet joint. The present invention provides devices and methods designed to aid in the correction, restoration or augmentation of target joint spaces, such as, facet joints at virtually all spinal levels including, but not limited to, L1-L2, L2-L3, L3-L4, L4-L5, L5-S1, T11-T12, and T12-L1.

One aspect of the invention provides, an implantable device that is placed through a joint space or joint complex, such that a central flexible section reinforces, replaces or augments the joint. The device can be delivered to the joint by access through bone into the joint space without opening or disrupting the joint space. The device reinforces, replaces or augments the joint complex including all or some of the capsule, ligaments, nucleus or other joint complex structures. The flexible central section acts as a flexible and/or conformable spacer with or without providing a fixed axis of rotation. Altering the flexibility of the flexible section can increase or decrease the constraint of the joint. Flexibility can easily be altered or revised in a subsequent procedure after initial implantation.

Another aspect of the invention provides, devices that allow placement of a device in a joint space without resection or compromising the capsule or surrounding tissue. The devices also allow variable distraction of two bony surfaces. Further the devices and methods do not rely on bony fixation to hold the device in place. However, the devices can use bony fixation, if desired. The devices allow for easy anatomical variations and a wide range of pathologies to be treated as the device contours itself to the surrounding structures. The device also enables a reduction in inventory for hospitals because one size can be adapted to fit many anatomical variations and pathologies.

Another aspect of the invention provides, an implantable artificial joint complex adapted for implantation within a target joint space within a human body comprising: an expandable joint segment adapted to fit within the target joint space; and a cannulated anchor adapted to engage the expandable joint segment and adapted to engage a bony structure adjacent the target joint space. The joint complex is suitable for use with a variety of joints, including the facet joint. The expandable joint segment may be variably expandable and may be formed from shape memory material. Additionally, the expandable joint segment may be coated with material that provides bony in-growth, or it may provide external teeth or anchors that engage the joint surface. In some instances, it may be desirable to remove all or part of the capsule surrounding the joint, in which case, the expandable joint segment may be expandable beyond the perimeter of the joint surfaces. In this case, the expandable joint segment forms a spacer between the joint surfaces and a capsule surrounding at least a part of the joint.

In some embodiments, the artificial joint complex the expandable joint segment is adapted to provide a low profile suitable for insertion through an access lumen that accesses a target joint space, such as a minimally invasive lumen formed in the bone. A second, larger profile, is achieved when the expandable segment is inflated while postioned within the lumen of the joint space. In other embodiments, the cannulated anchor is formed integrally with the expandable joint segment, while in still other embodiments, the cannulated anchor is removably connected. A cap for sealing the artificial joint complex is provided to seal the complex once installed and the expandable joint segment has been inflated. In some embodiments, a post, which can be centrally positioned, is positioned within any or all of the cannulated anchor or expandable joint segment. In some embodiments, it is contemplated that the cannulated anchor is a superior cannulated anchor that is adapted to engage a superior articular facet. In other embodiments, the cannulated anchor is an inferior cannulated anchor adapted to engage an inferior articular facet. In either of these embodiments, additional embodiments could provide a second anchor. Where a second anchor is provided, it could be either inferior to the superior cannulated anchor or superior to the inferior cannulated anchor. The second anchor, as with the first anchor, can be cannulated, if desired. Any of the embodiments can provide for the anchors to be threaded, either internally, externally, or both, to achieve the objectives of the design. Alternatively, the anchors could have a smooth exterior surface, a roughened exterior surface, or a coated exterior surface, as desired. The anchors could also be configured to deliver a target agent, such as a pharmaceutical or biological agent.

In some embodiments, it may be desirable to have flexibility of the anchor relative to the expandable joint segment. In such an embodiment, the cannulated anchor can be configured, for example, to provide a ball race within a lumen that engages a post communicating with the expandable joint segment positioned within the lumen of the anchor. In other embodiments, the anchors are cannulated with a post positioned within a lumen.

In some embodiments, the second expandable joint segment is adapted to fit within the target joint space and engages the second anchor. In those embodiments, a second expandable joint segment can be provided that is adapted to fit within the target joint space and engage the second anchor. However, in some embodiments, both the first and second expandable joint segments may be adapted to engage a single cannulated anchor. In either configuration, once expanded, the expandable joint segments can be configured to expand adjacent each other within the target joint space, or expanded such that one expanded segment fits within the other expanded segment, among other configurations.

Another aspect of the invention comprises an implantable artificial joint complex adapted for implantation within a target joint space within a human body comprising: an expandable joint segment adapted to fit within the target joint space; a first cannulated anchor adapted to engage the expandable joint segment and adapted to engage a bony structure adjacent the target joint space; and a second anchor adapted to engage the expandable joint segment and adapted to engage a bony structure adjacent a target joint space. In some embodiments, the expandable joint segment is variably expandable. In yet other embodiments, the expandable joint segment is formed from a shape memory material. In still other embodiments, the expandable joint segment is coated with a material that promotes bony in growth.

In some embodiments, the expandable joint segment is expandable beyond the perimeter of all, or a part, of the joint surfaces. Thus, the expandable joint segment can form a spacer between at least part of the joint surfaces as well as a capsule surrounding at least part of the joint. Additionally, the expandable segment can expand around the joint in such a manner than axial movement of the joint surfaces away from each other is restricted or prevented. The expandable joint segment is typically configured to provide a low profile for insertion through an access lumen, and a larger profile when inflated, such as when it is within the target joint space.

In some embodiments, the cannulated anchor is formed integrally with the expandable joint segment. In other embodiments, the cannulated anchor is removably connected to the expandable joint segment. In either case, a cap is provided to seal the artificial joint complex after the lumen of the expandable joint segment has been inflated. A post or reinforcement member can be provided within a lumen of the expandable joint segment and/or within the cannulated anchor.

When implanted, the artificial joint complex can comprise a first cannulated anchor that is a superior cannulated anchor adapted to engage a superior articular facet. Additionally, a second anchor, which can also be cannulated if desired, can be provided inferior to the superior cannulated anchor. Alternatively, the first cannulated anchor can be configured to be an inferior cannulated anchor adapted to engage an inferior articular facet. In that embodiment, the second anchor, which can also be cannulated if desired, can be provided superior to the inferior cannulated anchor. In any of these embodiments the first cannulated anchor and/or the second anchor can be interiorly or exteriorly threaded, as needed to provide anchoring or to engage a post or reinforcement member.

In some embodiments, the artificial joint complex can be configured to surround a post. In other embodiments, any anchor can be configured to provide a ball race within a lumen that engages a post also positioned within the lumen. Thus, the post can moveably engage the ball race of the cannulated anchor.

In other embodiments, exterior of the anchors and/or joint complex can include an exterior surface treatment to promote bony in-growth. In other embodiments, it may be desirable to provide a first and second expandable joint segment that are adjacent each other. In other embodiments, one of the first or second expandable joint segments can be configured to fit within another joint segment, such that, for example, the first joint segment or space fits with the second joint segment or spacer. The joint segments can each be inflatable from an anchor which is cannulated to allow administration of material that inflates the expandable joint segment. For example, the first joint segment is inflatable from a first anchor, while the second joint segment is inflatable from a second anchor. Alternatively, the first and second anchors could be inflatable from a single anchor. In this embodiment, the device could still be adapted to provide that a second anchor engage at least one of the first and second joint segments. However, that anchor need not be cannulated.

Another aspect of the invention comprises a method of implanting a patient specific artificial joint complex comprising: accessing a target joint space by creating an access hole through an adjacent bony structure; inserting a joint complex device having a cannulated anchor and an expandable joint segment through the access hole with the expandable joint segment being positioned between the surfaces forming the joint; injecting material into the expandable joint segment; and sealing access to the target joint space. In some aspects of the method, additional steps are provided, including one or more of: removing cartilage in the target joint space, resurfacing a joint surface in the target joint space and/or removing a capsule surrounding the target joint space. In some instances it may be desirable to revise the original implant, in which case, the expandable joint segment is re-accessed, such as through the cannulated anchor, and additional material is injected into the expandable joint segment, or material with withdrawn from the expandable joint segment. Additionally, the inflation material can be completely removed and replaced, if desired.

Yet another aspect of the invention comprises a method of implanting a patient specific artificial joint complex comprising: accessing a target joint space by creating an access hole through an adjacent bony structure; inserting a joint complex device having a cannulated anchor formed from biodegradable material and an expandable joint segment through the access hole with the expendable joint segment being positioned between the surfaces forming the joint; injecting material into the expandable joint segment; sealing access to the target joint space; and allowing the cannulated anchor to degrade in situ.

Still another aspect of the invention comprises a method of implanting a patient specific artificial joint complex comprising: accessing a target joint space by creating an access hole through an adjacent bony structure; inserting a cannulated injector device through the access hole with openings communicating with the target joint space; injecting material into the joint space; withdrawing the cannulated injector; and sealing access to the target joint space.

Another aspect of the invention comprises a device for creating a patient specific artificial joint complex comprising: a cannulated injector tube adapted to traverse an access lumen to a target joint space through a bony structure having an opening for communicating with the target joint space; and a removable flange connected to the cannulated injector tube and adapted to seal the access lumen upon removal from the target joint space.

Still another aspect of the invention comprises a kit or system for repairing, restoring or augmenting a joint surface. The kit comprises one or more cannulated and non-cannulated anchors that are adapted to securely engage an inflatable spacer or artificial joint segment, one or more inflatable spacers are provided to be used to complete the system before implantation.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a lateral view of a normal human spinal column;

FIG. 2 is a superior view of a normal human lumbar vertebra;

FIG. 3 is a lateral view of a functional spinal unit;

FIG. 4 is a postero-lateral oblique view of a vertebrae;

FIG. 5 is a perspective view of the anatomical planes of the human body shown in relation to a depiction of the human body;

FIG. 6 is a perspective view of the L4-L5 region of the lumbar spine illustrating selected ligaments and the articular capsule associated with a spinal facet joint;

FIG. 7A is a perspective side view of an embodiment of an artificial joint complex; FIG. 7B is a perspective side view showing the internal portions of the device in phantom; FIG. 7C is a cross-section of the device taken along the lines c-c of FIG. 7A; FIG. 7D is a cross-section of the device taken along the lines d-d of FIG. 7A; FIG. 7E is a cross-section of the device taken along the lines e-e of FIG. 7A; FIG. 7F is a perspective side view of the device of FIG. 7A in a deployed condition;

FIG. 8A is a perspective side view of an embodiment of an implantable artificial joint complex; FIG. 8B is a perspective side view showing the internal portions of the device in phantom; FIG. 8C is a cross-section of the device taken along the lines c-c of FIG. 8A; FIG. 8D is a cross-section of the device taken along the lines d-d of FIG. 8A; FIG. 8E is a cross-section of the device taken along the lines e-e of FIG. 8A; FIG. 8F is a perspective side view of the device of FIG. 8A in a deployed condition;

FIG. 9A is a perspective side view of an embodiment of an artificial joint complex; FIG. 9B is a perspective side view showing the internal portions of the device in phantom; FIG. 9C is a cross-section of the device taken along the lines c-c of FIG. 9A; FIG. 9D is a cross-section of the device taken along the lines d-d of FIG. 9A; FIG. 9E is a cross- section of the device taken along the lines e-e of FIG. 9A; FIG. 9F is a perspective side view of the device of FIG. 9A in a deployed condition;

FIG. 10A is a perspective side view of an embodiment of an implantable joint complex; FIG. 10B is a perspective side view showing the internal portions of the device in phantom; FIG. 10C is a cross-section of the device taken along the lines c-c of FIG. 10A; FIGS. 10D, 10D(1), 10D(2), 10D(3) and 10D(4) are cross-sectional views of the device taken along the lines d-d of FIG. 10A; FIG. 10E is a cross-section of the device taken along the lines e-e of FIG. 10A; FIG. 10F, 10F(1) and 10F(2) are perspective side view of the device of FIG. 10A in a deployed condition;

FIG. 11A is a side view of an embodiment of an artificial joint complex; FIG. 11B is a side view showing the internal portions of the device in phantom; FIG. 11C is a cross-section of the device taken along the lines c-c of FIG. 11A; FIG. 11D is a cross-section of the device taken along the lines d-d of FIG. 11A; FIG. 11E is a side view of the device of FIG. 11A in a deployed condition;

FIG. 12 is a cross-sectional view of an installed artificial joint complex implanted in a facet joint of a first embodiment;

FIG. 13 is a cross-sectional view of an installed artificial joint complex implanted in a facet joint of another embodiment;

FIG. 14 is a cross-sectional view of an installed artificial joint complex implanted in a facet joint of yet another embodiment;

FIG. 15 is a cross-sectional view of an installed artificial joint complex implanted in a facet joint of still another embodiment;

FIG. 16 is a cross-sectional view of an installed artificial joint complex implanted in a facet joint of yet another embodiment;

FIG. 17A-B is a cross-sectional view of an installed artificial joint complex implanted in a facet joint of another embodiment;

FIGS. 18A-B are perspective views of an installed artificial joint complex implanted in a facet joint of a functional spine unit; and

FIG. 19 illustrates a flow chart of a method for deploying an implantable joint complex or system of the invention.

### DETAILED DESCRIPTION OF THE INVENTION

The invention relates to implantable devices, including implantable prosthesis suitable for implantation within the body to restore, reinforce, replace and/or augment connective tissue such as bone, and systems and methods for treating spinal pathologies. The invention relates generally to implantable devices and apparatuses or mechanisms that are suitable for implantation within a human body to restore, augment, and/or replace soft tissue and connective tissue, including bone and cartilage, and systems for treating spinal pathologies. In various embodiments, the implantable devices can include devices designed to replace missing, removed or resected body parts or structure. The implantable devices, apparatus or mechanisms are configured such that the devices can be formed from parts, elements or components which alone, or in combination, comprise the device. Thus, for example, the implantable devices can be configured such that one or more elements or components are formed integrally to achieve a desired physiological, operational or functional result such that the components complete the device. Functional results can include the surgical restoration of the joint, restoration of the functional power of a joint, controlling, limiting or altering the functional power of a joint, and/or eliminating the functional power of a joint by preventing joint motion. Portions of the device can be configured to replace or augment existing anatomy and/or implanted devices, and/or be used in combination with resection or removal of existing anatomical structure. The device and its operation can be revised subsequent to the initial implantation, removed, or the inflation material can be changed (e.g. to convert the device from a spacer to one which promotes fusion of the joint).

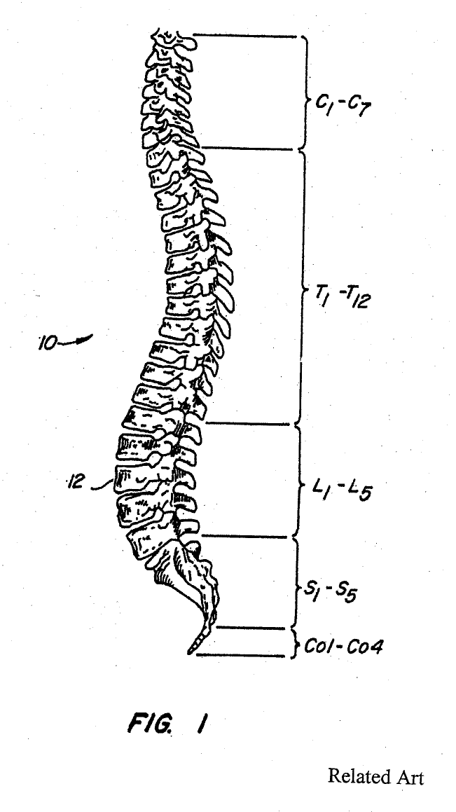

The implantable devices of the invention are designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4.

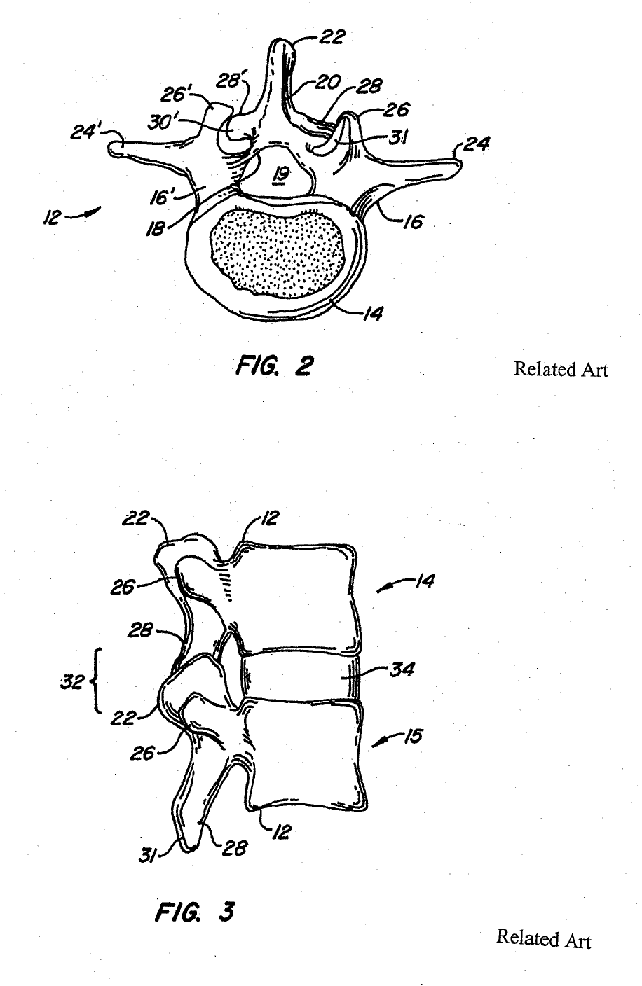

An example of one vertebra is illustrated in FIG. 2 which depicts a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles 16, 16′, extend dorsally from each side of the vertebral body 14 to form a vertebral arch 18 which defines the vertebral foramen 19.

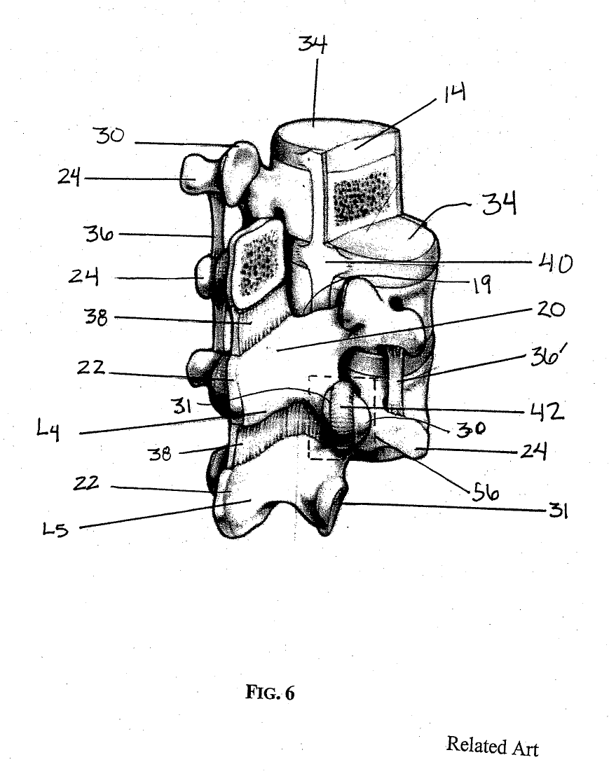

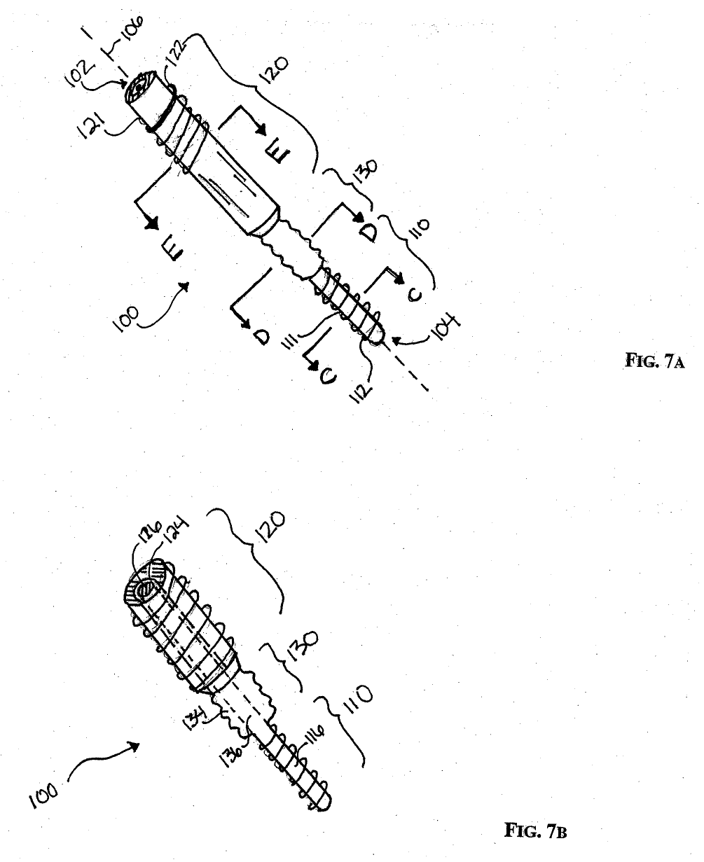

At the posterior end of each pedicle 16, the vertebral arch 18 flares out into broad plates of bone known as the laminae 20. The laminae 20 fuse with each other to form a spinous process 22. The spinous process 22 provides for muscle and ligamentous attachment as shown in FIG. 6. A smooth transition from the pedicles 16 to the laminae 20 is interrupted by the formation of a series of processes.

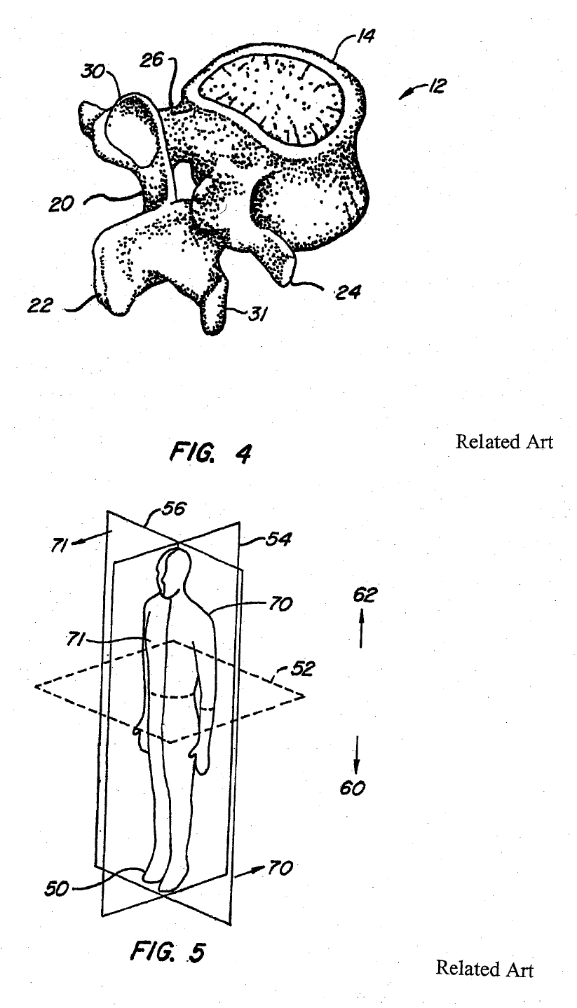

Two transverse processes 24,24′ thrust out laterally, one on each side, from the junction of the pedicle 16 with the lamina 20. The transverse processes 24,24′ serve as levers for the attachment of muscles to the vertebrae 12. Four articular processes, two superior 26, 26′ and two inferior 28, 28′, also rise from the junctions of the pedicles 16 and the laminae 20. The superior articular processes 26, 26′ are sharp oval plates of bone rising upward on each side of the vertebrae, while the inferior articular processes 28, 28′ are oval plates of bone that jut downward on each side. See also FIG. 4.

The superior and inferior articular processes 26 and 28 each have a natural bony structure known as a facet. The superior articular facet 30 faces medially upward, while the inferior articular facet 31 (see FIG. 3) faces laterally downward. When adjacent vertebrae 12 are aligned, the facets 30 and 31, capped with a smooth articular cartilage and encapsulated by ligaments, interlock to form a facet joint. The facet joints are apophyseal joints that have a loose capsule and a synovial lining.

As discussed, the facet joint 32 is composed of a superior articular facet 30 and an inferior articular facet 31 (shown in FIG. 4). The superior articular facet is formed by the vertebral level below the facet joint 32, and the inferior articular facet is formed in the vertebral level above the facet joint 32. For example, in the L4-L5 facet joint shown in FIG. 3, the superior articular facet of the facet joint 32 is formed by bony structure on the L5 vertebra (i.e., a superior articular surface and supporting bone 26 on the L5 vertebra), and the inferior articular facet of the facet joint 32 is formed by bony structure on the L4 vertebra (i.e., an inferior articular surface and supporting bone 28 on the L4 vertebra). The angle formed by a facet joint located between a superior articular facet and an inferior articular facet changes with respect to the midline of the spine 10 (see FIG. 1) depending upon the location of the vertebral body 14 along the spine 10 (e.g., cervical, thoracic, lumbar). The facet joints do not, in and of themselves, substantially support axial loads unless the spine 10 is in an extension posture (lordosis). As would be appreciated by those of skill in the art, the orientation of the facet joint 32 for a particular pair of vertebral bodies changes significantly from the thoracic to the lumbar spine to accommodate a joint's ability to resist flexion-extension, lateral bending, and rotation.

An intervertebral disc 34 between each adjacent vertebra 12 (with stacked vertebral bodies shown as 14, 15 in FIG. 3) permits gliding movement between each vertebra 12. The structure and alignment of the vertebrae 12 thus permit a range of movement of the vertebrae 12 relative to each other. FIG. 4 illustrates a posterolateral oblique view of a vertebra 12, further illustrating the curved surface of the superior articular facet 30 and the protruding structure of the inferior articular facet 31 adapted to mate with an opposing superior articular facet. As discussed above, the position of the inferior articular facet 31 and superior articular facet 30 varies on a particular vertebral body 14 to achieve the desired biomechanical behavior of a region of the spine.

Thus, the overall spine 10 comprises a series of functional spinal units that are a motion segment consisting of two adjacent vertebral bodies 14, 15, the intervertebral disc 34, associated ligaments, and facet joints 32. See, Posner, I, et al. “A biomechanical analysis of the clinical stability of the lumbar and lumbrosacral spine.” Spine 7:374-389 (1982).

As previously described, a natural facet joint, such as facet joint 32 (FIG. 3), has a superior articular facet 30 and an inferior articular facet 31. In anatomical terms, the superior articular facet of the joint is formed by the vertebral level below the joint, which can thus be called the “caudad” portion of the facet joint because it is anatomically closer to the tail bone or feet of the person and faces downward 60. The inferior articular facet of the facet joint is formed by the vertebral level above the joint, which can be called the “cephalad” portion of the facet joint because it is anatomically closer to the head of the person and faces upward 62. Thus, a device that, in use, replaces the caudad portion of a natural facet joint (i e., the superior articular facet 30) can be referred to as a “caudad” device. Likewise, a device that, in use, replaces the cephalad portion of a natural facet joint (i.e., the inferior articular facet 31) can be referred to a “cephalad” device.

When the processes, e.g. superior articular process 26 and inferior articular process 28, on one side of a vertebral body 14 are spaced differently from corresponding processes on the other side of the same vertebral body, components of the devices of the invention on each side would desirably be of differing sizes as well to account for anatomical difference that can occur between patients. Moreover, it can be difficult for a surgeon to determine the precise size and/or shape necessary for an implantable device until the surgical site has actually been prepared for receiving the device. In such case, the surgeon typically can quickly deploy a family of devices or components possessing differing sizes and/or shapes during the surgery. Thus, embodiments of the devices of the present invention include modular designs that are either or both configurable and adaptable. Additionally, the various embodiments disclosed herein may also be formed into a kit or system of modular components that can be assembled in situ to create a patient specific implant. As will be appreciated by those of skill in the art, as imaging technology improves, and mechanisms for interpreting the images (e.g., software tools) improve, patient specific designs employing these concepts may be configured or manufactured prior to the surgery. Thus, it is within the scope of the invention to provide for patient specific devices with integrally formed components that enable the device to act in a uniform manner and that are pre-configured. Further, the practice of the present invention can employ, when necessary to practice the invention, conventional methods of x-ray imaging and processing, x-ray tomosynthesis, ultrasound including A-scan, B-scan and C-scan, computed tomography (CT scan), magnetic resonance imaging (MRI), optical coherence tomography, single photon emission tomography (SPECT) and positron emission tomography (PET) within the skill of the art. Such techniques are explained fully in the literature and need not be described herein. See, e.g., Essentials of Radiologic Science, Fosbinder and Kelsey, 2002, The McGraw-Hill Companies, publisher; X-Ray Structure Determination: A Practical Guide, 2nd Edition, editors Stout and Jensen, 1989, John Wiley & Sons, publisher; Body CT: A Practical Approach, editor Slone, 1999, McGraw-Hill publisher; X-ray Diagnosis: A Physician's Approach, editor Lam, 1998 Springer-Verlag, publisher.

A configurable modular device design, such as the devices enabled by this invention, allows for individual components to be selected from a range of different sizes and utilized within a modular device. One example of size is to provide inferior and superior stems or rods of various lengths. The stems or rods form permanent or semi-permanent (e.g., where bioresorbably material is used) anchors for the spacer that forms the artificial joint segment. The stems or rods can be cannulated, as necessary or desirable, to provide a mechanism for filling the spacer with material. A modular implantable device design allows for individual components to be selected for different functional characteristics as well. The components can, provide connections sized to communication with other components, or adaptors (not shown) can be provided to connect one component to another. One example of function is to provide stems having different surface features and/or textures to provide anti-rotation capability. Other examples of the configurability of modular implantable device of the present invention as described in greater detail below.

Implantable devices can be configurable such that the resulting implantable device is selected and positioned to conform to a specific anatomy or desired surgical outcome. The adaptable aspect of device provides the surgeon with customization options during an implantation or revision procedure. It is the adaptability of the device systems that also provides adjustment of the components during the implantation procedure to ensure optimal conformity to the desired anatomical orientation or surgical outcome. An adaptable modular device allows for the adjustment of various component-to-component relationships. Configurability may be thought of as the selection of a particular size of component that together with other component size selections results in a custom fit implantable device. Adaptability then can refer to the implantation and adjustment of the individual components within a range of positions in such a way as to fine tune the “custom fit” devices for an individual patient. The net result is that embodiments of the modular, configurable, adaptable spinal device and systems of the present invention allow the surgeon to alter the size, orientation, and relationship between the various components of the device to fit the particular needs of a patient during the actual surgical procedure.

In order to understand the configurability, adaptability, and operational aspects of the invention, it is helpful to understand the anatomical references of a human body 50 with respect to which the position and operation of the devices, and components thereof, are described. There are three anatomical planes generally used in anatomy to describe the human body and structure within the human body: the axial plane 52, the sagittal plane 54 and the coronal plane 56 (see FIG. 5). Additionally, devices and the operation of devices are better understood with respect to the caudad 60 direction and/or the cephalad direction 62. Devices positioned within the body can be positioned dorsally 70 (or posteriorly) such that the placement or operation of the device is toward the back or rear of the body. Alternatively, devices can be positioned ventrally 71 (or anteriorly) such that the placement or operation of the device is toward the front of the body. Various embodiments of the joint complexes and systems of the present invention may be configurable and variable with respect to a single anatomical plane or with respect to two or more anatomical planes. For example, a component or device may be described as lying within and/or having adaptability or operability in relation to a single plane.

FIG. 6 is a perspective view of the L4-L5 region of the lumbar spine illustrating ligaments and the articular capsule associated with a spinal facet joint. Three stacked vertebral bodies 14 are depicted. The superior vertebral body 14 has been cut open to illustrate the interior of the vertebral body 14 and the superior surface of the intervertebral disc 34. Between each of the transverse processes 24 in an adjacent pair of vertebra 12 is an intertransverse ligament 36, 36′ connecting the two transverse processes on each side of the spine. Similarly, between each pair of adjacent spinous processes 22 is an interspinous ligament 38. A posterior longitunal ligament 40 runs along the length of the spine within the vertebral foramen 19 adjacent the surface of the vertebral body 14 defining the vertebral foramen 19. As discussed above, the facet joint 32 is located where the superior articular facet 30 and the protruding structure of the inferior articular facet 31 mate. The surface of the facet joint 32 is covered with an articular capsule 42.

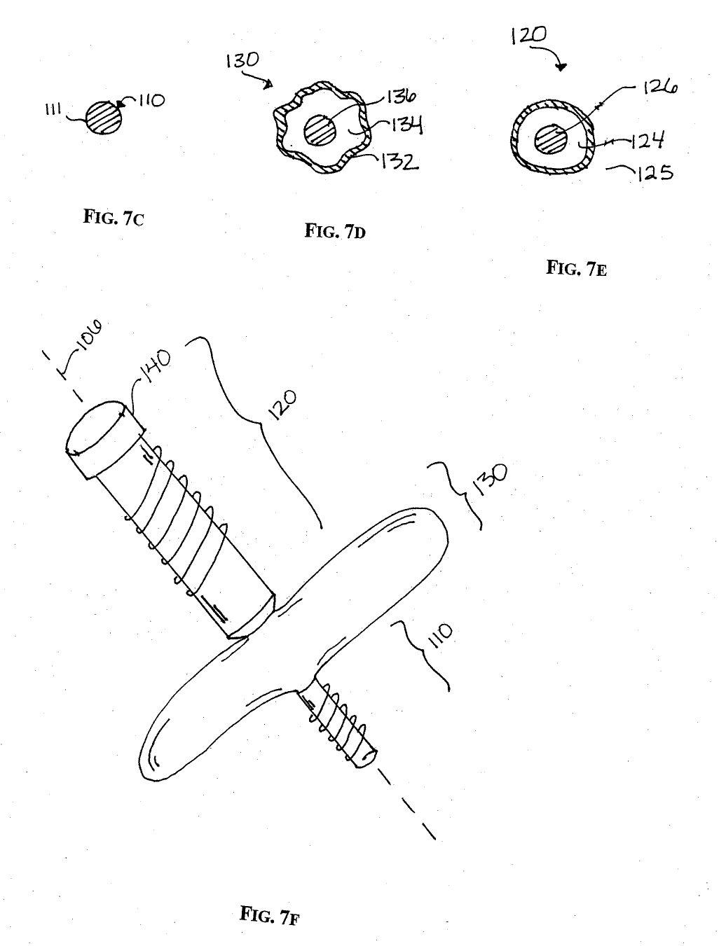

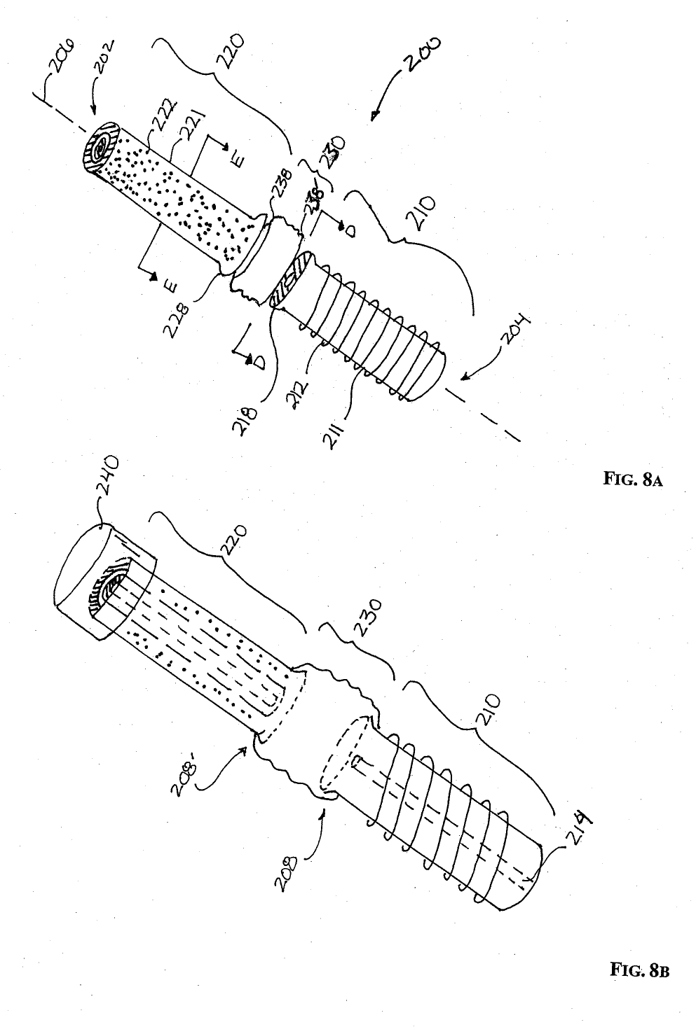

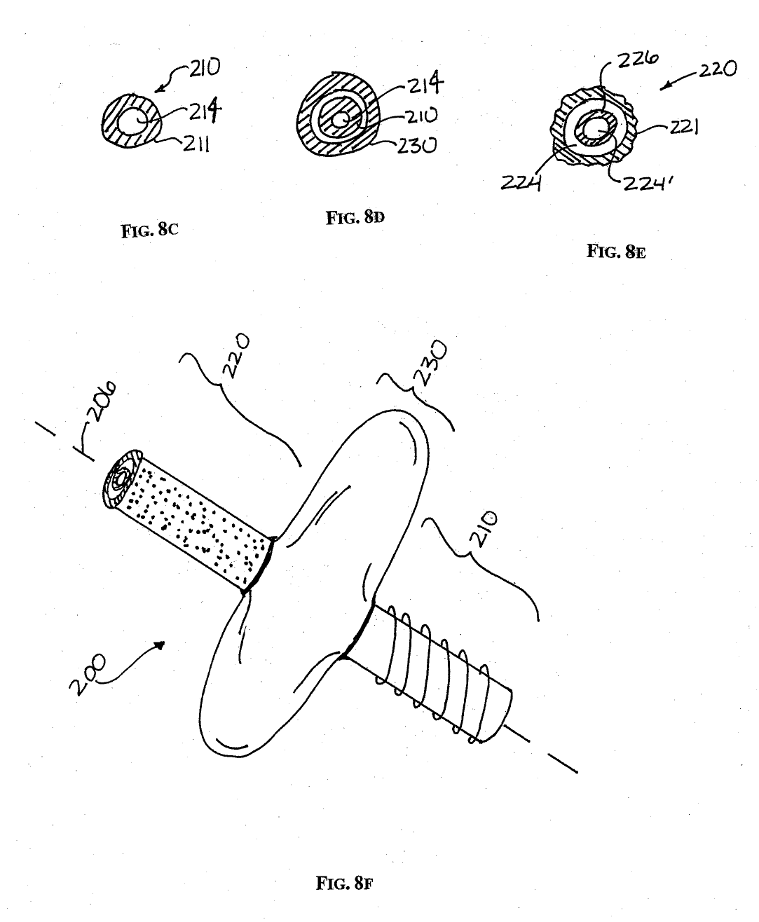

Turning now to FIG. 7A, a side view of an embodiment of a joint complex 100 is depicted. The joint complex 100 has an elongated profile with a first or inferior end 102 and a second or superior end 104. The device 100 is oriented and operates with respect to an axis 106, which may or may not be central along the entire length of the device. A superior or cephalad post or rod 110, or support structure, is positioned at one end and an inferior or caudad post or rod 120, or support structure, is positioned at an opposing end. Positioned between the superior post or anchor 110 and the inferior post or anchor 120 is a variably expandable joint segment 130 or spacer. The variably expandable joint segment 130 can be formed as an inflatable balloon or fabric weave pouch which is delivered to the cavity or space of the target joint, i.e. the space between the two joint surfaces. The components 110, 120, 130 can be formed integrally such that they are manufactured as a single piece or such that the pieces act in a unified manner. Alternatively, it may be desirable to form the joint complex 100 as a series of components, such as components that can be selected and sized to treat a particular patient's anatomical disease. As illustrated in FIG. 7A either or both (as illustrated) of the superior anchor 110 and the inferior anchor 120 can be configured to provide a threaded 112, 122 exterior surface 111, 121 to facilitate anchoring the superior anchor 110 and inferior anchor 120 within an aperture formed in the respective inferior and superior surfaces of, for example, the facet joint 32 (see, FIGS. 3 and 6). The threads 112, 122 can be configured with different pitches to allow compression or distraction. The threads can be provided exteriorly (as shown) and/or interiorly (not shown) to enable the device 100 to securely mate with the bone and to enable components of the device 100 to securely mate with each other, e.g. a post located within a lumen formed in another post, such as the inferior or superior anchor.

FIG. 7B is a side view showing the internal portions of the device 100 in phantom. As shown in this view, the inferior anchor 120 is cannulated to provide a lumen 124 that enables the variably expandable joint segment 130 to be filled with suitable material to expand the artificial joint 130 or spacer within the space between the two joint surfaces such as facet joint surfaces 30, 31 (see, FIGS. 3 and 6). A post 116 can be provided that extends through any or all of the superior anchor 110, the inferior anchor 120 and the variably expandable joint segment 130 which defines a lumen 134 configured for expansion. As illustrated in FIG. 7B the post 116 extends through the superior anchor 110 and the variably expandable joint segment 130 to provide stability to the device 100. As will be appreciated, the post 116 can be configured to be positioned within the superior anchor 110 and the variably expandable joint segment 130 and then exit the variably expandable joint segment 130 to form the superior anchor 110. The post can also have a three part configuration such that the superior anchor 116 connects to an intermediate post 136 which, in turn, connects to an inferior anchor 126. Other variations to the configuration could be made without departing from the scope of the invention.

FIG. 7C is a cross-section of the device 100 taken along the lines c-c of FIG. 7A. In the embodiment shown, the superior anchor 110 is a solid post with an exterior surface 111. However, as will be appreciation, the configuration of the post could take a variety of forms without departing from the scope of the invention, including forming a hollow tube with or without threads on the exterior surface to engage the bone. FIG. 7D is a cross-section of the device 100 taken along the lines d-d of FIG. 7A. The variably expandable joint segment 130 has an exterior component 132 configured such that it can achieve a low profile to facilitate deployment within a facet joint space through an access aperture, but with enough elasticity that the layer 132 of the variably expandable joint segment 130 can be expanded by filling its lumen 134 with a suitable material to a greater profile that adapts to the contours of the facet joint space. As will be appreciated by those of skill in the art, the lumen 134 can be evenly filled or variably filled, e.g., where the lumen comprises discrete compartments, with each compartment separately fillable by differing amounts, if desired. The central portion 136 of the variably expandable joint segment 130 can be an extension of the central post 116 or can be a separate post configured to mate with the central post 116 of the inferior anchor 110.

Turning now to FIG. 7E, a cross-section of the device taken along the lines e-e of FIG. 7A is depicted. The inferior anchor 120 has an exterior tube 125, which can be threaded as shown in FIGS. 7A-B, which is adapted to engage portions of the inferior articular process 28 and/or spinous process 22. The interior of the exterior tube 125 has one or more lumens 124 for communicating from an inferior end 102 of the device 100 to the expandable section of the variably expandable joint segment 130. The lumen 124 can be configured to circumnavigate a central post 126 provided in the interior of the exterior tube 125, as illustrated, or can be configured to provide access from the inferior end 102 to the expandable section by any other suitable configuration. Central post 126 can be provided, as shown, and be positioned to traverse the inferior anchor 110 section of the device 100. Central post 126 can provide many functions, including, for example, providing stability where the lumen 124 has been configured to circumnavigate the post.

FIG. 7F is a perspective side view of the device of FIG. 7A in a deployed condition. As evidenced from this figure, the variably expandable joint segment 130 expands radially away from the longitudinal central axis 106 of the device 100 upon inflation. In situ, this inflation enables the variably expandable joint segment 130 to fill a target joint space and accommodate any imperfections or irregularities to the facet joint anatomy and thus improve or restore function of the joint.

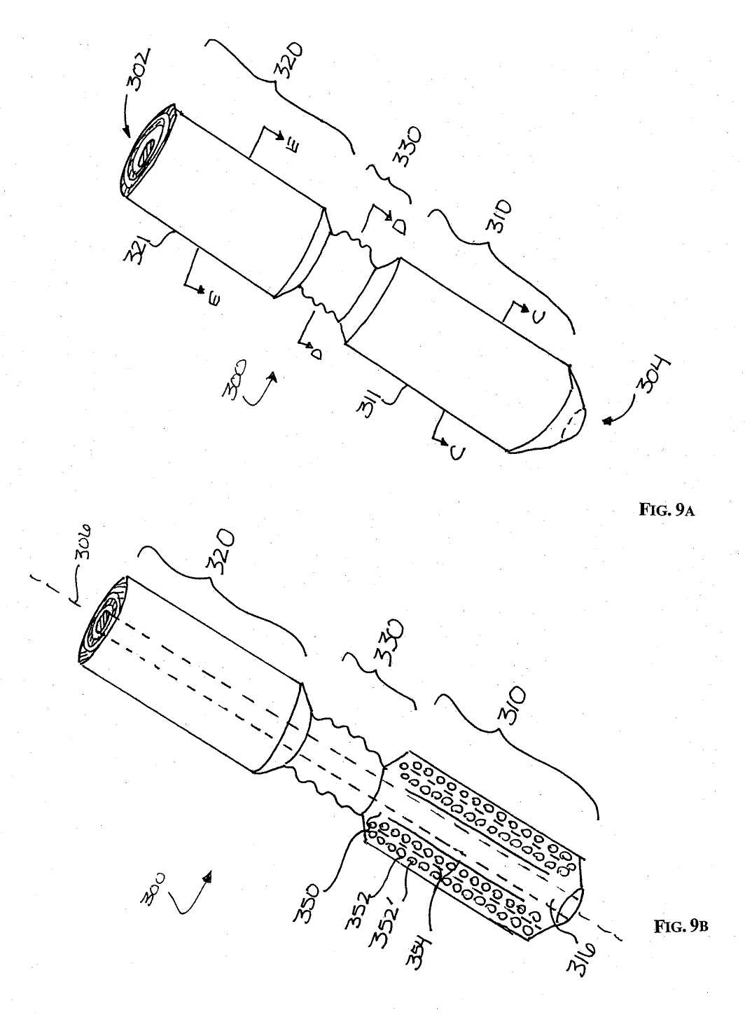

FIG. 8A is a perspective side view of an embodiment of a joint complex 200 in an exploded configuration. The device 200 has an inferior end 202 and a superior end 204. An inferior anchor 220, which can be in the form of a hollow or cannulated tube, terminates at the inferior end 202. In this configuration, the exterior surface 221 of the inferior anchor 220 is configured with a surface adapted to promote anchoring within a target bony structure, for example, by providing a roughened surface 222 or by providing an exterior coating that promotes the device being secured to the bony surface. The superior anchor 210 can likewise be cannulated or can be configured as a solid post or rod. As depicted, the exterior 211 of the superior anchor 210 has threads 212 which facilitates adapting the superior anchor 210 portion of the device 200 to engaging the target bony structure, such as the superior articular facet. An variably expandable joint segment 230 is provided between the inferior anchor 220 and the superior anchor 210. The variably expandable joint segment 230 is adapted in this embodiment to releasably engage both the inferior anchor 220 and the superior anchor 210 by, for example, providing a pair of flairs 238, 238′ that snap fit over a pair of flanges 218, 228 on each rod 210, 220. In an alternate embodiment, the variably expandable joint segment 230 could be configured to releasably engage only one of the inferior anchor 220 and the superior anchor 210. Alternatively, the anchors 210, 220 could be configured to snap fit over the joint segment 230.

FIG. 8B is a side view showing the internal portions of the device 200 in phantom. As depicted in this embodiment, superior anchor 210 is configured to provide a lumen 214. The lumen 214 can be configured to communicate with the variably expandable joint segment 230 or be sealed within the interior of the superior anchor 210. Providing a lumen 219 can be used where, for example, it is desirable that the superior anchor 210 have flexibility, or where it is desirable to fill the rod with material after insertion into the target bony structure of the spine. Other advantages of a lumen 219 would be apparent to those skilled in the art. The inferior anchor 220 can also be configured with a lumen 224 and a central post 216, if desired. The figure also depicts the junction 208′ at the superior end of the inferior anchor 220 and the junction 208 at the inferior end of the superior anchor 210.

FIG. 8C is a cross-section of the device 200 taken along the lines c-c of FIG. 8A. The lumen 219 is positioned centrally within the superior anchor 210 and the exterior surface 211 has a substantially circular shape with a smooth exterior surface at the cross-section c-c. FIG. 8D is a cross-section of the device taken along the lines d-d of FIG. 8A, which is approximately where the superior anchor 210 mates with the variably expandable joint segment 230. The lumen 214 fits within the superior end 208 of the variably expandable joint segment 230. As depicted, the inferior end of the superior anchor 210 can be configured to provide a flanged end 218 which releasably and securely engages an aperture of a variably expandable joint segment 230 adapted to mate with the flanged end 218. FIG. 8E is a cross-section of the device 200 taken along the lines e-e of FIG. 8A. The inferior anchor 220 in this embodiment is generally depicted as having a circular cross-section with an exterior surface 221 that is roughened to promote adhesion with the bony surface. The inferior anchor 220 has a lumen 224 extending therethrough that is defined by a central post 226 that extends along the interior of the inferior anchor 220. An additional lumen 224′, configured in this embodiment as a lumen within the post 226, can also be provided. The lumen can be adapted to provide access to the interior of the lumen 234 of the variably expandable joint segment 230.

FIG. 8F is a perspective side view of the device 200 of FIG. 8A in a deployed condition wherein the variably expandable joint segment 230 has been inflated or expanded.

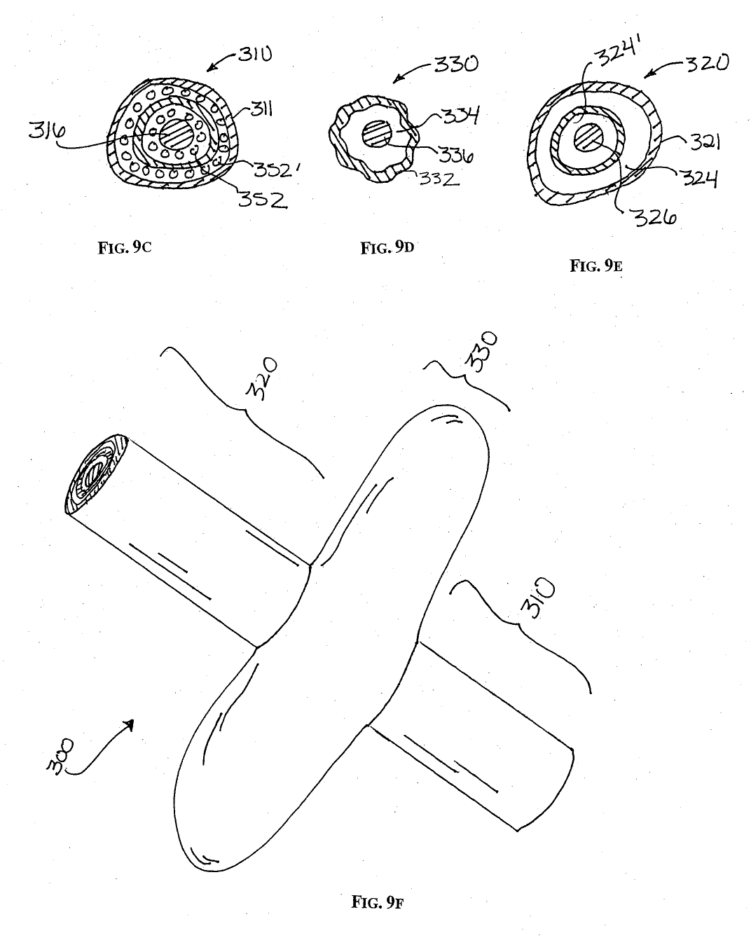

FIG. 9A is a perspective side view of yet another embodiment of a joint complex 300. In this embodiment, both the exterior surfaces 311, 321 of the superior anchor 310 and inferior anchor 320 are smooth. FIG. 9B is a perspective side view showing the internal portions of the device 300 in phantom. In this embodiment, the superior anchor 310 has been configured to provide a bearing assembly 350 within the lumen. The bearing assembly 350 enables additional flexibility between the superior anchor 310 and the variably expandable joint segment 330. As will be appreciated by those skilled in the art, the bearing assembly can be provided on either or both posts 310, 320, but for purposes of illustration has been depicted in conjunction with the superior anchor 310. The bearing assembly 350 includes a plurality of rolling bearing elements 352 and a retaining device 354. An inner and outer ring is formed by the balls on a track forming a ball race 355. The bearing elements 352 roll in grooves or tracks and enable movement between the post 316 and the ball race 355 allowing movement along an elongated central axis 306 of the post 316 relative to the superior anchor 310. FIG. 9C is a cross-section of the device 310 taken along the lines c-c of FIG. 9A. The device 300 has a post 316 positioned centrally within the device. In some embodiments, however, it may be desirable to position the post 316 off-centrally within the device 300. The bearing assembly 350 includes an inner and outer layer of bearing element 352, 352′ that slideably engage the post 316. An exterior tube 319 having a lumen sized to enable the ball race 355 to fit within the interior of the exterior tube 319 is provided. Turning now to FIG. 9D, a cross-section of the device 300 taken along the lines d-d of FIG. 9A illustrating a central post 336 located within a lumen 334 that, in this configuration, circumnavigates the post 336. The exterior layer 332 has a variable geometric configuration due to the inflatable nature of the variably expandable joint segment 330. The exterior layer 332 can be formed like an inflatable balloon that can be folded or reduced to a low profile in order to access the interior of the facet joint space using a hole (not shown) drilled in the bony structure that is sized to facilitate either or both of the superior anchor 310 or inferior anchor 320 traversing the hole. FIG. 9E is a cross-section of the device taken along the lines e-e of FIG. 9A. As depicted in FIG. 9E, two lumens 324, 324′ are provided along with a post 326 positioned centrally. FIG. 9F is a perspective side view of the device 300 of FIG. 9A in a deployed condition with the variably expandable joint segment 330 in an expanded position.

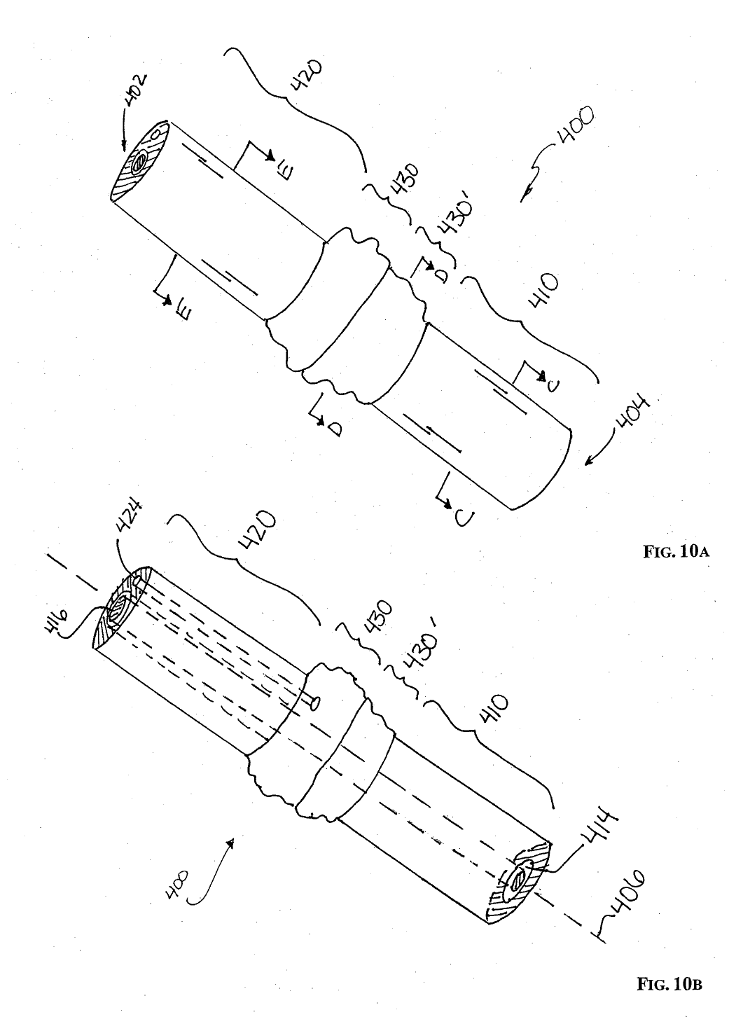

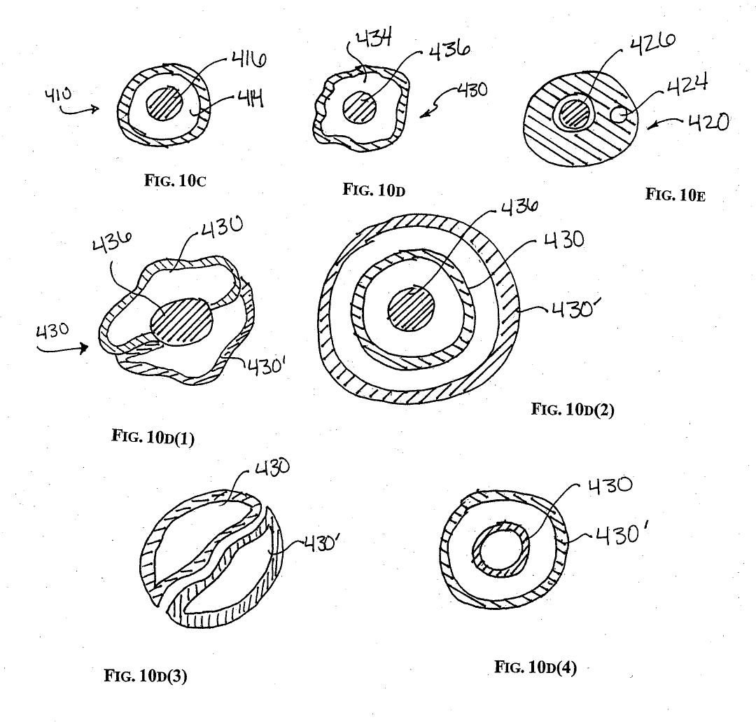

FIG. 10A is a perspective side view of yet another embodiment of a joint complex 400. In this embodiment, more than one variably expandable joint segment 430, 430′ is provided. As illustrated here, two variably expandable joint segments 430, 430′ are provided between the superior anchor 410 and the inferior anchor 420 in a stacked manner. These two sections can be snap fit between the superior anchor 410 and the inferior anchor 420, or can be formed integrally with one or more posts. Additionally, as will be appreciated by those skilled in the art, other embodiments could be configured, including but not limited to providing additional sections, if desired, or positioning the sections in a side-by-side configuration or an encapsulated configuration (where one of the variably expandable joint segments fits within the second variably expandable joint segment) without departing from the scope of the invention.

As illustrated in FIG. 10B, which is a side view showing the internal portions of the device 400 in phantom, the inferior anchor 410 is configured with a lumen 414 providing access to an variably expandable joint segment 430′ while the superior anchor 420 is configured with an off-center lumen 424 providing access to another variably expandable joint segment 430. A single integrated central post 416 has been provided that extends from the inferior anchor 410 through each of the variably expandable joint segments, where the variably expandable joint segments 430, 430′ are configured to positioned adjacent each other in a manner where each variably expandable joint segment is intersected by the elongate central axis 406. FIG. 10C is a cross-section of the device 400 taken along the lines c-c of FIG. 10A illustrating the lumen 414 and the central post 416. As will be appreciated by those of skill in the art, the lumen can have a circular cross-section, or an ovoid cross-section, for example where it is desirable for the variably expandable joint segment 430 to have lateral movement relative to the central axis 406 of the device. FIG. 10D is a cross-section of the device 400 taken along the lines d-d of FIG. 10A illustrating an artificial joint segment 430′ having a lumen 434. The post 436, in this embodiment, extends from the lumen 414 of the superior anchor 410 into the lumen 434 of the artificial joint segment 430′.

FIGS. 10D(1) and 10D(2) illustrate cross-sectional views for alternate embodiments having a post. For example, in the embodiment shown in FIG. 10D(1) the artificial joints segments 430, 430′ are configured to be surround the post 436 such that each artificial joint segment is positioned on a lateral portion of the post 436. each adjustable joint segment or spacer has a lumen. In contrast, FIG. 10D(2) illustrates an embodiment, where a first artificial joint segment or balloon 430 extends from, for example, the inferior anchor 420 and is adapted to fit within or be encapsulated by a second artificial joint segment or balloon 430′ that extends from, for example, the superior anchor 410. In contrast, FIGS. 10D(3) and 10D(4) illustrate still other embodiments of the device where a post 436 is not provided within the lumen of the artificial joint segment 430. As will be appreciated by those of skill in the art, the artificial joint segment 430, 430′ can be inflated or expanded to provide different pressures. Thus, for example, referring to FIG. 10D(4) in one embodiment, the centrally positioned artificial joint segment 430 could be expanded to a pressure that is higher than the pressure in the artificial joint segment 430′ that is configured to surround the joint segment 430. Such a configuration could provide variable support to the joint surfaces. Other configurations will be apparent to those skilled in the art.

FIG. 10E is a cross-section of the device 400 taken along the lines e-e of FIG. 10A illustrating the off-center lumen 424 of the inferior anchor 420 and post 426. FIG. 10F is a side view of the device of FIG. 10A having a first artificial joint segment 430 positioned adjacent a second artificial joint segment 430 such that both artificial joint segments are evenly bisected by a longitudinal central axis 406 in a deployed condition. FIGS. 10F(1) and 10F(2) illustrate additional deployed embodiments corresponding to the configuration of the artificial joint segments 430, 430′ described above with respect to FIGS. 10D(1) and 10D(2).

FIG. 11A is a side view of an embodiment of a joint complex 500. The joint complex 500 has an injector 560 and an inflatable or configurable artificial joint segment 530. FIG. 11B is a side view of the device 500 showing the internal portions in phantom. The injector 560 is configured to fit within the artificial joint segment 530. Prior to deployment, the complex 500 achieves a low cross-sectional profile allowing easy access into a target joint space. FIG. 11C is a cross-section of the device 500 taken along the lines c-c of FIG. 11A. The injector 560 has an interior lumen 562 through which material can be injected to access the interior of the artificial joint segment. FIG. 11D illustrates a cross-section of the device 500 taken along the lines d-d of FIG. 11A. In this view, the injector 560, having a lumen 562, fits within a lumen 534 of the artificial joint complex 530. FIG. 11E is a side view of the device of FIG. 11A in a deployed condition. The injector 560 is positioned within the lumen 534 of the artificial joint segment 530 which also has an injector tube 538 which is used to engage the injector 560 when filling the lumen 534. The injector tube 538 can, for example, be a semi-rigid tube capable of sealingly engaging the injector 560. The injector 560 has a generally radially extending flange 564, which appears similar to the head of a pin or nail, that can be shaped to have a t-shaped cross-section that enables it to engage the injector tube 538 to prevent complete removal of the injector 560 when filling is complete. Other configurations could be used without departing from the scope of the invention Thus, the radially extending flange 564 can act as a stopper to prevent the injector tube 560 from being completely pulled through the semi-rigid tube. Additionally, the flange 564 can contribute to the reliability of the seal. The injector 560 can be formed as a hollow passageway that is removably engageable with the flange 564 which may be adapted to engage a length of tube or a solid shaft. With either configuration, the injector portion that is a hollow passageway 566 is configured to have one or more openings 568 that, when the injector 560 is advanced within the lumen 534 of the facet joint 530 enable injectable material to pass through the hollow passageway 566, through the opening(s) 568 and into the lumen 534 of the artificial joint segment 530. When the injector 560 is withdrawn, the opening(s) 568 no longer communicate with the lumen 534 and the flange 564 abuts against the injector tube 538 sealing the apparatus. As described above, the injector 560 can be configured such that the flange 534, or the flange 534 in combination with a portion of post, is detachable along seam 569 from the injector 560 to leave the flange 534 in situ and to seal the artificial joint segment 530.

As will be appreciated by those of skill in the medical device and orthopedic arts, a variety of materials would be suitable for making the devices and the components of the devices described above. Suitable materials include, for example, biocompatible and biodegradable or bioresorbable materials known in the art. Biocompatible materials are typically those materials for which there is no medically unacceptable toxic or injurious effect on biological functions. As is known in the material sciences, assessing biocompatibility can include, for example, an assessment of nontoxicity and bioactivity as it relates to interacting with and, in time, being integrated into the biological environment, as well as other tailored properties that are desirable for a particular application. Suitable materials include those materials that restore and improve physiologic function, and enhance quality of life. Typically, the suitable materials fall into several categories including: inorganic materials (metals, ceramics, and glasses) and polymeric materials (synthetic and natural). Additionally, medical adhesives, dental composites, hydrogels, hyaluronic gels, and polymers for controlled slow drug delivery may also be suitable for use in the invention.

Suitable polymeric materials can be selected from a wide variety of known biocompatible and biodegradable polymers, such as those classified as polystyrenes, polyphosphoester, polyphosphazenes, aliphatic polyesters and their copolymers, such as polycaprolactone, hydroxybutyric acid, and butylenes succinate. Other polyesters, such as nylon, and natural polymers, such as modified polysaccharides, may also be appropriate, depending upon the application. In some instances, it may be desirable to use a shape memory polymer that has the ability to store and record large strains. Still other polymers include polyetherehterketone, polyetherketoneketone, polyethylene, fluoropolymers, elastomers and the like. Other appropriate polymers that can be used in the components or devices are described in the following documents, all of which are incorporated herein by reference: PCT Publication WO 02/02158 A1, dated Jan. 10, 2002 and entitled Bio-Compatible Polymeric Materials; PCT Publication WO 02/00275 A1, dated Jan. 3, 2002 and entitled Bio-Compatible Polymeric Materials; and PCT Publication WO 02/00270 A1, dated Jan. 3, 2002 and entitled Bio-Compatible Polymeric Materials. Still other materials such as Bionate®, polycarbonate urethane, available from the Polymer Technology Group, Berkeley, Calif., may also be appropriate because of the good oxidative stability, biocompatibility, mechanical strength and abrasion resistance. Combinations of any suitable material, including the materials listed here, can be used as well, without departing from the scope of the invention.

Thus, for example, the superior anchor and/or the inferior anchor of the devices of FIGS. 7-10 could be formed from suitable metals, such as titanium, cobalt chromium, and surgical stainless steel, a well as from suitable ceramics and polymeric materials. Shape memory metals, such as nitinol may also be desirable. Additionally, combinations of suitable materials can be used as desired. For example, an interior component, such as the central post, could be manufactured from one material while the exteriorly formed component could be formed of a suitable second material. Additionally, components could be coated with, for example, a suitable bioceramic or polymer to facilitate implantation.

Materials suitable for filling the variably expandable joint segment of the devices of FIGS. 7-10, or for use with the injector of the devices of FIG. 11, described above, include biocompatible polymers, biocompatible foams, such thermoplastic syntactic foam, water-insoluble derivatives of hyaluronic acid in the form of gels, films and sponges, polyglycolic acid, low-density reticulated vitreous carbon (RVC), and hydrogels. In some instances, the injectable material may be a gas. The materials can be prepared in colored form by including a dye or stain to assist in easier handling and visualization during or after the surgical process. The materials can also be selected for its ability to become more or less viscous as the material approaches body temperature, or to provide growth factors, antibiotics, or other agents to the site. Materials may also be loaded with pharmaceutical agents which are delivered to the site by a permeable or semi-permeable membrane. Additionally, materials that promote fusion of the joint, either initially or where the device is revising an originally implanted system, may also be used without departing from the scope of the invention.

In some instances, it may be desirable to form all, or a part of the devices in bioresorbable polymers. Bioresorbable materials are those materials made from essentially the same lactic acid molecular building blocks that occur naturally in the human body. Long polymer chains are created to form polylactides (PLa). Thus for example, a containment implant can be formed of biologically and biomechanically active PLa which is then resorbed during the healing process, leaving only the facet joint section implanted.

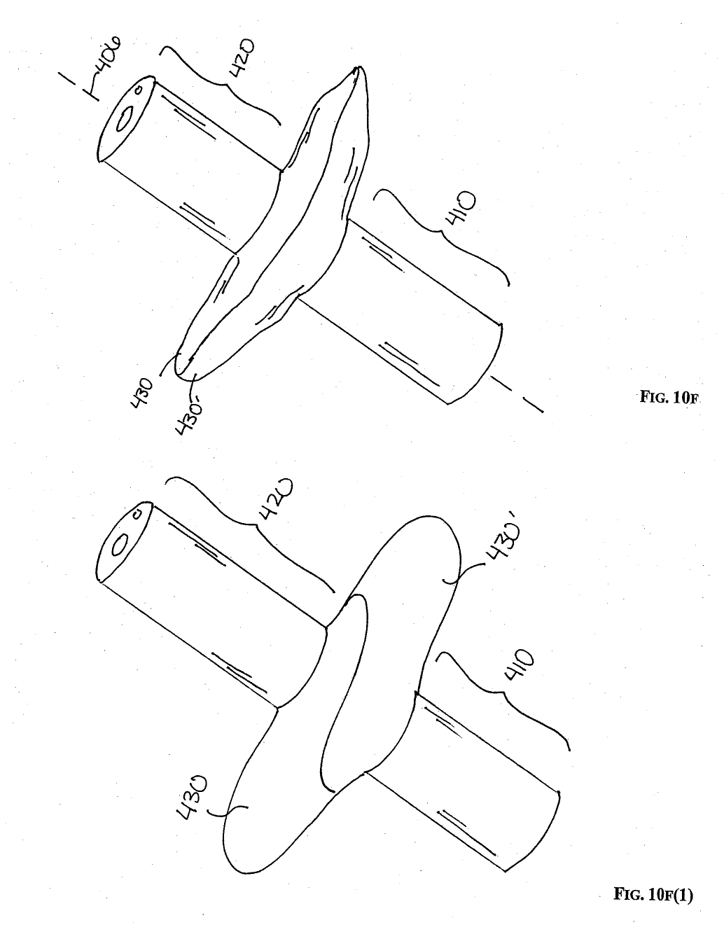

FIG. 12 is view of an installed joint complex 100 of a first embodiment implanted in a facet joint 32, such as the embodiment illustrated in FIG. 7. The cross-sectional view is depicted along a coronal, or nearly coronal, plane cut across the superior articular facet 30 and inferior articular facet 31 forming the facet joint 32, as indicated by the dashed lines 56 in FIG. 6. As depicted in FIG. 12, the articular capsule 42 is intact, or largely intact, and the interior space of the facet joint 32 has been accessed through an aperture 80. The surfaces of the facet joint 32 can optionally be modified to, for example, remove any cartilage, such as damaged cartilage and/or burrs to the bony surface, e.g. by smoothing or conforming the joint surface. In this embodiment, the installed joint complex 100 is depicted as only partially advancing through the bone of the superior articular facet 30. As will be appreciated by those skilled in the art, the installed facet joint complex 100 can advance through the entire superior articular facet 30 such that it extends out the opposing end where it can be optionally engaged by a cap or nut to anchor and/or seal the device.

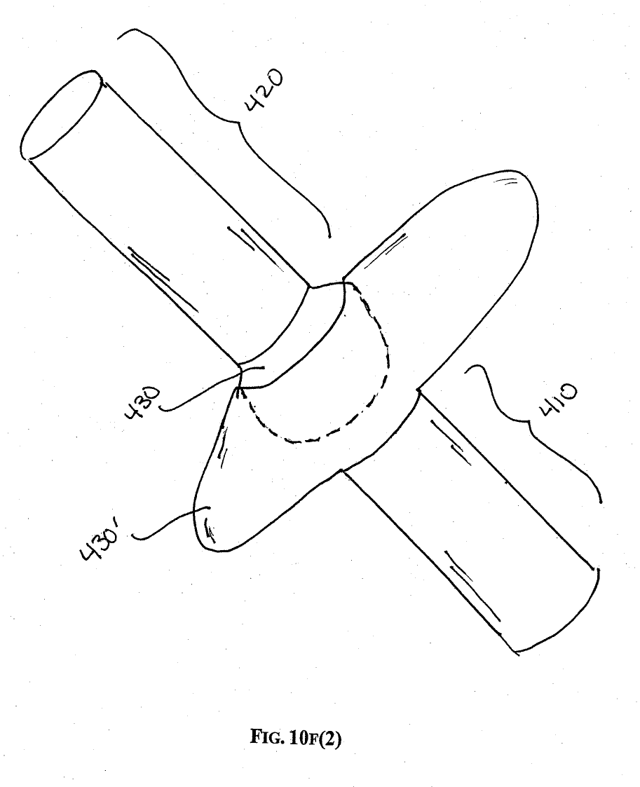

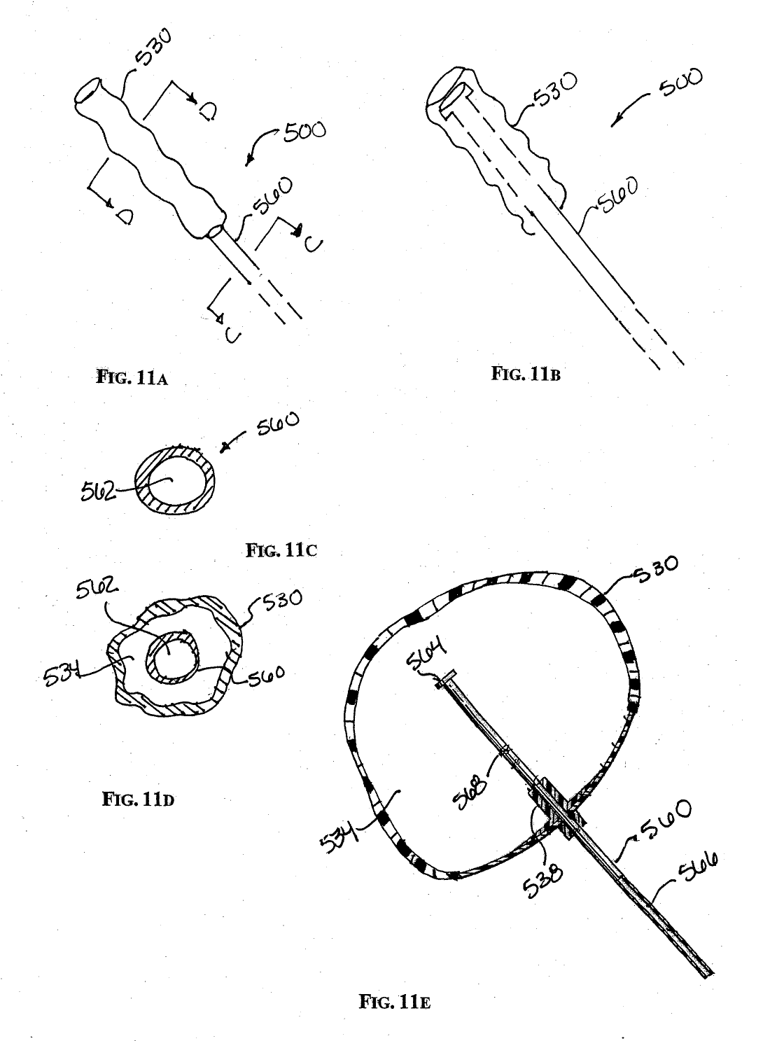

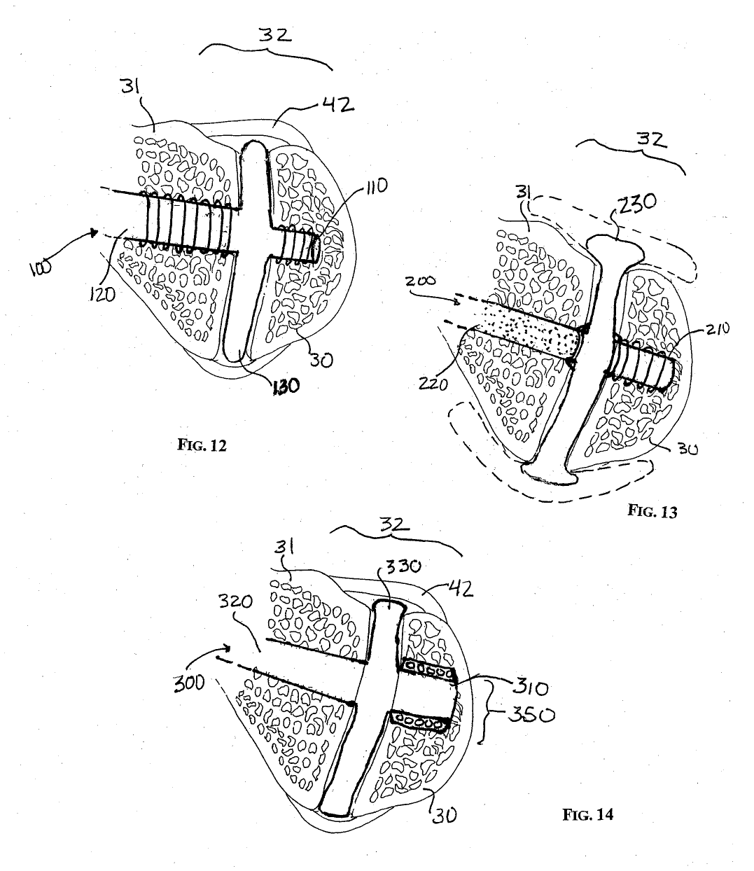

FIG. 13 is view of another installed joint complex 200 implanted in a facet joint such as that depicted in FIG. 8 above. In this cross-sectional view, the articular capsule 42 of (see FIG. 6) has been removed to allow the inflated artificial joint spacer 230 to expand beyond the lateral edges of the facet joint 32. Where the articular capsule 42 has been removed, it may be desirable to inflate the artificial facet joint 230 so that it extends beyond the lateral edges of the facet joint and acts as replacement for the articular capsule 42 by encircling the facet joint 32 in a manner similar to the natural articular capsule 42, or in a manner that restores part of the missing articular capsule, as well as mechanical support for the facet joint 32. As illustrated by the dashed lines, the expansion of the spacer 230 can be such that it captures the joint and prevents movement of the joint surfaces away from each other by wrapping around bony protuberances. As will be appreciated by those of skill in the art, this configuration can be used with a two joint system, such as that shown in FIG. 10.

FIG. 14 is view of another installed joint complex 300 such as that depicted in FIG. 9 above which has been installed in a facet joint 32. In this embodiment, the bearing assembly 350 is depicted associated with one end of the complex 300 and enabling at least one end of the complex 300 to moveably engage the inflated artificial facet joint 330. In this embodiment, the articular capsule 42 has been depicted as intact. However, as will be appreciated by those of skill in the art, the articular capsule need not be intact.

FIG. 15 is view of an installed joint complex 400 of yet another embodiment illustrated implanted within a facet joint 32, similar to that depicted in FIG. 10 above. In this embodiment, two inflated artificial joints 430, 430′ are depicted adjacent one another. As described above, however, alternate configurations of the artificial joints 430, 430′ can be employed without departing from the scope of the invention.

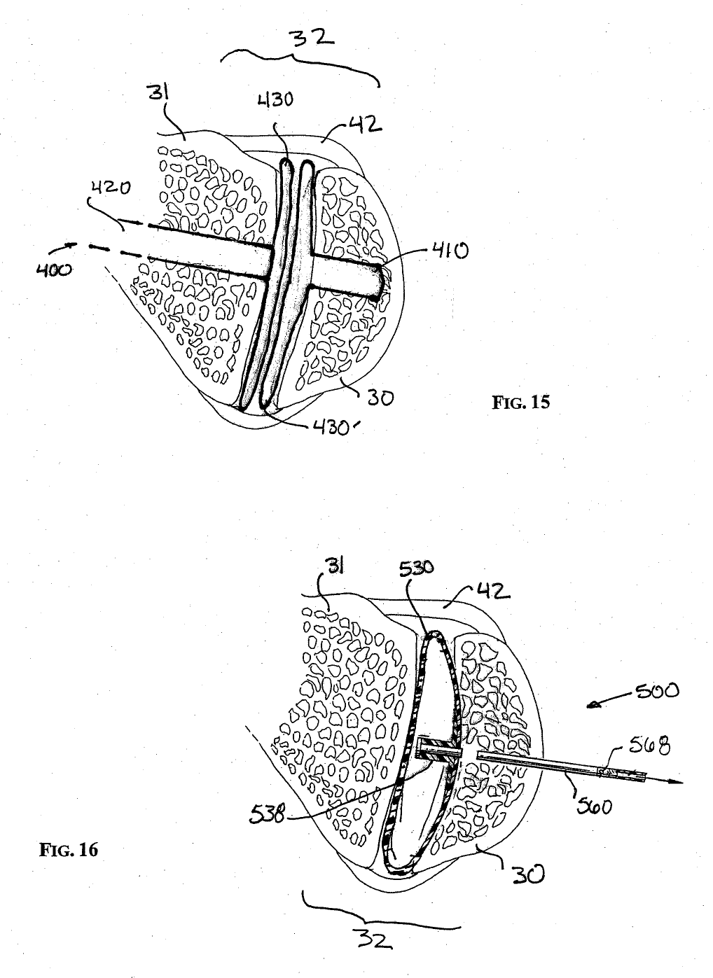

FIG. 16 is view of an installed joint complex 500 of still another embodiment illustrated implanted within a facet joint 32, similar to that depicted in FIG. 11 above. In this embodiment, the artificial joint segment 530 is positioned within an intact articular capsule 42. The artificial joint is accessed by a single injection device 560, which, when withdrawn, provides a self-sealing function, as illustrated.

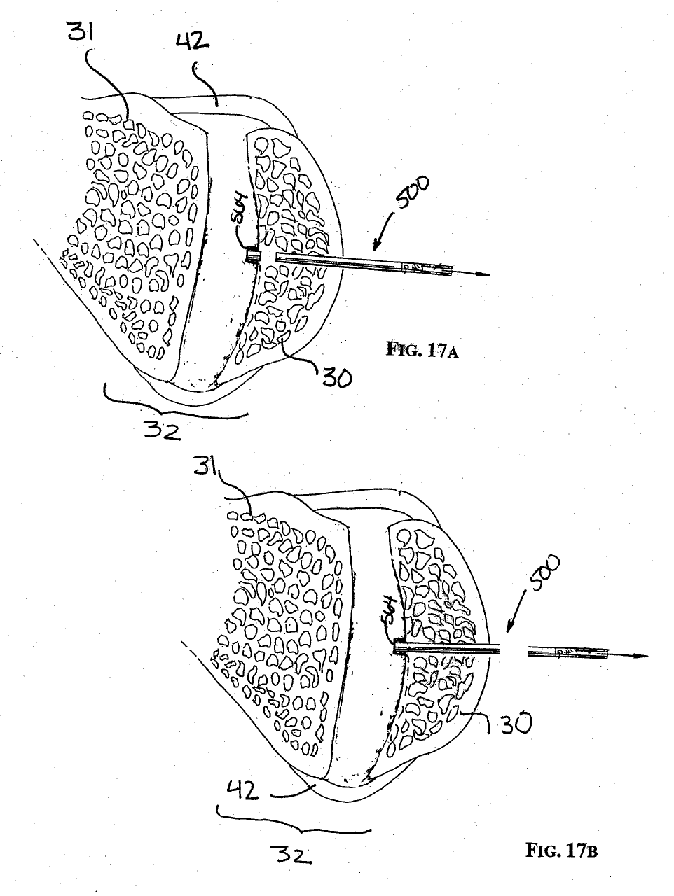

FIGS. 17A-B are views of systems 600 for achieving a joint complex of still another embodiment of the invention. In this embodiment, the articular capsule 42 is intact and provides the exterior dimension of the artificial joint which is formed by injecting foam or gel within the natural space defined by the two joint surfaces and surrounded by the articular capsule.

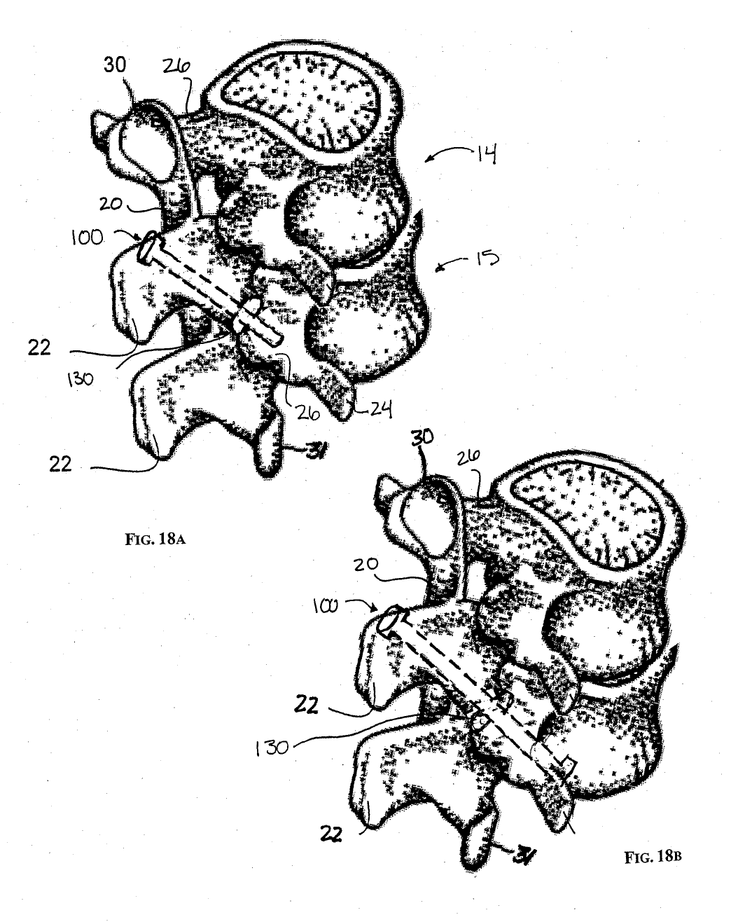

FIGS. 18A-B illustrate perspective views of two vertebral bodies 14,15 of a spinal column having an installed facet joint complex of any of a number of the embodiments depicted above. In FIG. 18A the inferior anchor extends into but not through the inferior articular process 28 and the spinous process 22 of the cephalad vertebra, and the superior anchor extends into, but does not extend through, the superior articular process 26 of the caudad vertebra. In FIG. 18B the inferior anchor extends into and through the inferior articular process 28 of the cephalad vertebra, and the superior anchor extends through, the superior articular process 26 of the caudad vertebra. As will be appreciated by those skilled in the art, the depiction of the operation and function of the devices described above with respect to the inferior articular process 28 and superior articular process 26 is made for purposes of illustration. A reverse configuration or operation relative to the inferior articular process 28 and the superior articular process 26 can be made without departing from the scope of the invention.

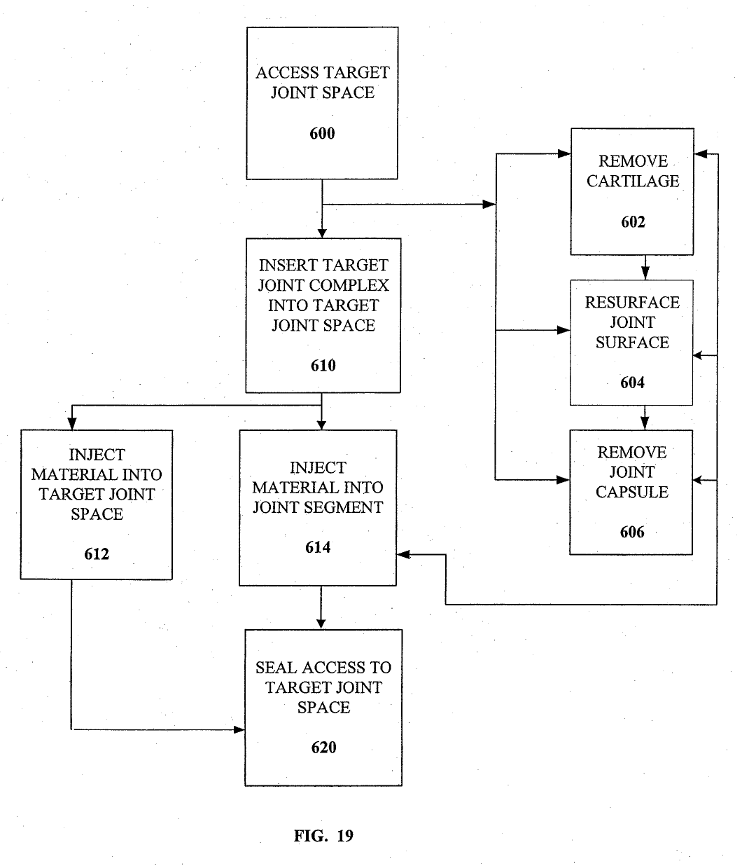

FIG. 19 illustrates a flow chart for a process of deploying a device of an embodiment of the invention. The steps of implanting the devices described above, include accessing the target joint space 600. Accessing the joint space is typically accomplished using minimally invasive techniques, such as by providing a bore hole through a section of bone to access the joint space. Prior to inserting the inflatable joint segment, it may be desirable to remove cartilage 602, resurface the joint surface 604, and/or remove the capsule 606. Either after the optional steps that attend to the joint surface, or after creating an access hole, the joint complex is inserted into the target joint space 610. As will be appreciated by those of skill in the art, the joint complexes described above can be inserted such that device crosses the entire joint surface as well as the surrounding bones, or can pass through a part of the bone surfaces. Typically, at least one bone surface is crossed in order to create an access lumen. The opposing bone surface may not be breached, or may be partially engaged, or fully engaged by a post or anchor of the device. Once the joint complex in inserted into the target joint space 610, material can be injected into the target joint space 612 or material can be injected into the expandable joint segment 614. Once a desired amount of material has been injected into the space, the access to the target joint space is sealed 620. As will be appreciated by those of skill in the art, this process can be revised to increase the amount of injectable material provided, or to decrease the amount of injectable material provided, as desirable or necessary. Additionally, the device can be withdrawn entirely by extracting the material injection and withdrawing the device through the minimally invasive lumen created for its insertion.

Once implanted, the device can be held in place in a variety of ways, depending upon how the invention has been practiced. For example, the joint section, can be attached to one or more cannulated fixation pins such as those used to install the joint section. Alternatively, the joint section can become ingrown into resected bone. In yet another alternative, the joint section can be contained within the natural articular capsule, which is fully or partially intact. Further, the joint section can wrap around prominences in the bone upon inflation where the articular capsule is completely or partially missing. Finally, the joint section can be provided with spikes, or attachment points, that penetrate the surrounding bone upon inflation to secure the device within the joint space.

After the device is installed in the facet joint of the spine, if there is further movement of the vertebral bodies 34 relative to each other, the device can be accessed again following the same steps and procedures described above, and the inflation of the device can be changed to effectively relocate the vertebral bodies.

The method and devices of the invention described above are also suitable for use in other applications within the body. For example, the small joints of the finger or toes, as well as the ankle. The device has been described in terms of implantation within the facet joint of a spine for purposes of illustration. As will be appreciated, the device and method could be used for other joint surfaces, such as those in the hand, feet, ankle and elbow, without departing from the scope of the invention.

While preferred embodiments of the invention have been illustrated and described, it will be appreciated that various changes can be made therein without departing from the spirit and scope of the invention. Moreover, while the present inventions have been described for use with a modular artificial joint system, it should be understood that the present inventions have utility in conjunction with the measurement and placement of other artificial joint systems, including single component, multi-component and custom-made artificial joints, with varying results. Further, the trialing system described herein can comprise single or multi-component tools and devices.

1. A facet joint implant that can treat ailments of the spine, the implant comprising: a facet joint spacer adapted to be inserted into a facet joint; an anchoring plate extending from the facet joint spacer and adapted to be attached to the spine; and said facet joint spacer including an inferior shim and a superior shim, wherein said inferior shim is stiffer and less compliant that the superior shim.

2. The implant of claim 1 wherein said facet joint spacer is secured to the anchoring plate with an articulation joint.

3. The implant of claim 1 wherein said superior shim is molded onto said inferior shim.

4. The implant of claim 1 wherein said inferior shim defines an inferior surface of the facet joint spacer and at last one protrusion extends from said inferior surface.

5. The implant of claim 1 wherein said inferior shim defines an inferior surface of the facet joint spacer and at least one protrusion extends from said inferior surface which said protrusion is comprised of a metal.

6. The implant of claim 1 wherein said superior shim is secured to the inferior shim.

7. A facet joint implant that can treat ailments of the spine, the implant comprising: a facet joint spacer adapted to be inserted into a facet joint; an anchoring plate extending from the facet joint spacer and adapted to be attached to the spine; and said facet joint spacer including an inferior shim and a superior shim, wherein said inferior shim is comprised of a different material than the superior shim.

8. The implant of claim 1 wherein said facet joint spacer is secured to the anchoring plate.

9. The implant of claim 1 wherein said inferior shim defines an inferior surface of the artificial facet joint and at least one protrusion extends from said inferior surface.

10. The implant of claim 1 wherein said inferior shim is secured to the inferior shim.
